(19) United States
(12) Patent Application Publication
(10) Pub. No.: US 2006/0275780 A1
(43) Pub. Date: Dec. 7, 2006

(54) Cross-linking reagents and uses thereof

(75) Inventors: Robert S. Matson, Orange, CA (US); Raymond Milton, La Habra, CA (US)

Correspondence Address: HOGAN & HARTSON L.L.P., 1999 AVENUE OF THE STARS, SUITE 1400, LOS ANGELES, CA, 90067, US

(73) Assignee: Beckman Coulter, Inc.

(21) Appl. No.: 11/145,670

(22) Filed: Jun. 6, 2005

(51) Int. Cl. C12Q 1/68; C12M 1/34; C07H 21/04

(52) U.S. Cl. 435/6; 435/287.2; 427/2.11; 536/25.32

(57) ABSTRACT

A method of cross-linking a polynucleotide to a target compound. The method comprises (1) providing a polynucleotide and a target compound, and (2) contacting the polynucleotide and the target compound with a photoreactive cross-linking agent under conditions that allow cross-linking of the polynucleotide to the target compound. Also disclosed is a kit comprising a polynucleotide and a photoreactive cross-linking agent for cross-linking the polynucleotide to a target compound.

### FIELD OF THE INVENTION

The present invention generally relates to cross-linking reagents and methods. Specifically, the invention relates to use of photoreactive agents to cross-link polynucleotides to target compounds such as proteins.

### BACKGROUND OF THE INVENTION

Cross-linking is a useful technique for studying multi-molecular complexes. SomaLogic has demonstrated photo-induced cross-linking of 5-bromo-2-deoxyuridine (BrdU) aptamers to their cognitive protein ligands (Bock et al., 2004, Proteomics 4:609-618). The method requires use of either an excimer laser or a high intensity UV lamp source. Both of these systems are expensive and difficult to automate. In addition, synthesis, purification and testing of BrdU oligonucleotides are costly and time-consuming. Recently, Fancy and Kodadek (1999, Proc. Natl. Acad. Sci. USA 96(11):6020-4) showed that protein-protein cross-linking can be achieved through photolysis of a ruthenium complex in the presence of ammonium persulfate (APS) and a weak intensity white light source. However, there remains a need for a rapid and efficient method for cross-linking a polynucleotide to a target compound.

### SUMMARY OF THE INVENTION

The present invention provides a simple, rapid and inexpensive means for cross-linking a polynucleotide to a target molecule. In particular, the method described in detail below is an attractive alternative to the laser-mediated cross-linking of BrdU-derivatized aptamer oligonucleotides (photo-aptamers) to their cognitive proteins. The present invention also provides a kit for cross-linking a polynucleotide to a target molecule using such a method.

In one aspect, the invention features a method of cross-linking a polynucleotide to a target compound. The method involves (1) providing a polynucleotide and a target compound, and (2) contacting the polynucleotide and the target compound with a photoreactive cross-linking agent under conditions that allow cross-linking of the polynucleotide to the target compound.

The polynucleotide is as an electron donor that donates electrons to the target compound, an electron acceptor. The polynucleotide may be a naturally occurring, synthetic, or modified molecule. For example, the polynucleotide may be a modified electron donor which contains one or more BrdU bases. The target compounds include polynucleotides, polypeptides, and other biomolecules, as well as their derivatives. For example, the target compound may be a modified electron acceptor created by modifying the target compound with an electron acceptor such as a poly-tyrosine or other phenolic polymers. As used herein, a “photoreactive cross-linking agent” refers to a chemical species that catalyzes or participates in a cross-linking reaction in the presence of light. In one embodiment, the cross-linking agent is activated by a visible light. A “visible light” is an electromagnetic radiation at a wavelength which a human eye can see (from ˜400 nanometers to ˜700 nanometers). In another embodiment, Ru(II)bpy₃²⁺ is used as the photoreactive cross-linking agent to cross-link a polynucleotide to a target compound in the presence of APS.

Another aspect of the present invention provides a method of identifying a target compound that binds a compound. The method involves (1) providing a compound and a test compound, either of which is a polynucleotide (2) contacting the compound and the test compound with a photoreactive cross-linking agent in a system, and (3) determining whether the compound is cross-linked to the test compound. If the compound is cross-linked to the test compound, the test compound is identified as a target compound that binds the compound. In one embodiment, either the compound or the test compound is a polypeptide. In another embodiment, both the compound and the test compound are polynucleotides.

More specifically, the invention provides a method of identifying a target compound that binds a polynucleotide (e.g., where the polynucleotide is known and the target compound is unknown). The method involves (1) providing a polynucleotide and a test compound, (2) contacting the polynucleotide and the test compound with a photoreactive cross-linking agent in a system, and (3) determining whether the polynucleotide is cross-linked to the test compound. If the polynucleotide is cross-linked to the test compound, the test compound is identified as a target compound that binds the polynucleotide. The present invention also provides a method of identifying a target polynucleotide that binds a compound (e.g., where the compound is known and the target polypeptide is unknown). The method involves (1) providing a compound and a test polynucleotide, (2) contacting the compound and the test polynucleotide with a photoreactive cross-linking agent in a system, and (3) determining whether the compound is cross-linked to the test polynucleotide. If the compound is cross-linked to the test polynucleotide, the test polynucleotide is identified as a target polynucleotide that binds the compound.

These methods can be carried out in cell-based systems or cell-free systems, where the polynucleotides or the compounds may be immobilized to substrates such as microtiter plates or beads.

For the methods described above, the cross-linked polynucleotide-target compound or compound-target compound can be analyzed by affinity interaction, size exclusion, electrophoresis, or a combination thereof. For example, the affinity interaction may be an immunoaffinity capture process, an immunoaffinity hybridization process, a nucleic acid or aptamer microarray-based hybridization process, or a combination thereof. Specifically, the immunoaffinity capture process may comprise antibody, antigen, aptamer probe arrays, immunoaffinity chromatography, or a combination thereof. The size exclusion may be gel chromatography, high performance size exclusion chromatography, membrane-based size exclusion, or a combination thereof. The electrophoresis is gel electrophoresis, capillary electrophoresis, capillary affinity interaction electrophoresis, or a combination thereof. Additionally, the target compound and the polynucleotide of the cross-linked polynucleotide-target compound or the target compound and the compound of the cross-linked compound-target compound may be identified by mass spectrometry.

The invention also provides a method for attaching a polynucleotide to a substrate. The steps of the method include (1) providing a polynucleotide, (2) providing a substrate containing a compound that binds the polynucleotide, and (3) contacting the polynucleotide and the compound with a photoreactive cross-linking agent under conditions that allow cross-linking of the polynucleotide to the compound. For example, the compound may be a polypeptide and the substrate may be a microtiter plate or a bead such as a magnetic bead.

Another aspect of the invention is directed to a kit for carrying out the cross-linking method described above. The kit contains a polynucleotide and a photoreactive cross-linking agent for cross-linking the polynucleotide to a target compound. Again, the polynucleotide may be a naturally occurring, synthetic, or modified molecule (e.g., BrdU-modified molecule). The target compounds include polynucleotides, polypeptides, and other biomolecules, as well as their derivatives. The photoreactive cross-linking agent (e.g., Ru(II)bpy₃²⁺) is capable of being activated by a light source (e.g., a visible light). When Ru(II)bpy₃²⁺ is used as the photoreactive cross-linking agent, a polynucleotide can be cross-linked to a target compound in the presence of APS.

The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1 is a photograph of a 6% urea gel showing SYBR Gold staining and silver staining of the bFGF photoaptamer-FGFb ligand complex.

FIG. 2 is a photograph of A² plates containing various aptamer-FGFb samples.

FIG. 3 demonstrates binding and cross-linking of FGFb ligand (1:1000 dilution, starting concentration 18 uM) to bFGF photoaptamer (the upper panel) and bFGF native aptamer (the lower panel).

FIG. 4 illustrates titration of FGFb ligand with bFGF photoaptamer and bFGF native aptamer.

FIG. 5 shows binding curves of modified (bFGF photoaptamer, in diamonds) and unmodified (bFGF native aptamer, in squares) aptamers.

FIG. 6 is a schematic representation of proposed cross-linking reaction between a BrdU-modified aptamer and a protein.

### DETAILED DESCRIPTION OF THE INVENTION

The invention is based on an unexpected discovery that a polynucleotide can be cross-linked to a target compound by a photoreactive cross-linking agent. Specifically, as described in the examples below, bFGF photoaptamer and bFGF native aptamer (Smith et al., 2002, Mol. Cell. Proteomics 2.1; and Bock et al., 2004, Proteomics 4:609-618) were cross-linked to FGFb ligand using Ru(I)bpy₃²⁺ that is activated by a white light in the presence of APS.

Fancy and Kodadek showed that protein-protein cross-links can be rapidly and efficiently achieved using photolysis of a ruthenium complex in the presence of ammonium persulfate (Fancy and Kodadek, 1999, Proc. Natl. Acad. Sci. USA 96(11):6020-4). The protein-protein cross-linking relies upon the presence of tyrosine residues within close proximity to each other in the paired proteins, i.e., the proteins must interact with one another. While the building blocks of proteins are amino acids, polynucleotides are comprised of nucleotides which are chemically and structurally distinct from amino acids. As polynucleotides do not have tyrosine residues, it was surprising to find that a polynucleotide can be cross-linked to a target compound, e.g., a polypeptide, by a photoreactive cross-linking agent. Without binding by the theory, it is believed that other electron donors such as thymidine might participate in the reaction provided that a protein-nucleic acid binding event were possible in order to bring, e.g., protein-tyrosine in close proximity to a nucleic acid-thymine. Thus, nucleic acid binding proteins and aptamers are primary target candidates.

Accordingly, the invention features a method of cross-linking a polynucleotide to a target compound. The method comprises the steps of (1) providing a polynucleotide and a target compound, and (2) contacting the polynucleotide and the target compound with a photoreactive cross-linking agent under conditions that allow cross-linking of the polynucleotide to the target compound.

As used herein, the term “polynucleotide” refers to a polymer of deoxyribonucleotides or ribonucleotides, in the form of a separate fragment or as a component of a larger construction. A polynucleotide may be a DNA, an RNA, a DNA analog such as PNA (peptide nucleic acid) and BrdU-DNA, or a synthesized oligonucleotide. The DNA may be a single- or double-strand DNA, or a DNA amplified by PCR. The RNA may be an mRNA or siRNA (small interfering RNA). In certain embodiments, the sequences of the polynucleotides are random, or may be generated from libraries of random DNA sequences. In other embodiments, the sequences of the polynucleotides may not be random, but rather be designed to react with specific target compounds. In particular, the polynucleotides may be aptamers (see, e.g., U.S. Pat. Nos. 5,270,163, 5,567,588, 5,650,275, 5,670,637, 5,683,867, 5,696,249, 5,789,157, 5,843,653, 5,864,026, 5,989,823, and WO 99/31275).

A polynucleotide molecule can be modified at the base moiety, sugar moiety or phosphate backbone to improve, e.g., the stability, binding ability, or solubility of the molecule. For non-limiting examples of synthetic polynucleotides with modifications, see Toulme, 2001, Nature Biotech. 19:17 and Faria et al., 2001, Nature Biotech. 19:40-4. For example, the deoxyribose phosphate backbone of the polynucleotide molecules can be modified to generate peptide nucleic acids (see Hyrup et al., 1996, Bioorganic & Medicinal Chemistry 4:5-23). A “peptide nucleic acid” or “PNA” is a nucleic acid analog, e.g., a DNA analog, in which the deoxyribose phosphate backbone is replaced by a pseudopeptide backbone and only the four natural nucleobases are retained. The neutral backbone of a PNA allows for specific hybridization to DNA and RNA under conditions of low ionic strength. The synthesis of PNA oligomers can be performed using standard solid phase peptide synthesis protocols as described in Hyrup et al., 1996, supra and Perry-O'Keefe et al., 1996, Proc. Natl. Acad. Sci. USA 93:14670-5.

In other embodiments, the polynucleotides may include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6; Lemaitre et al., 1987, Proc. Natl. Acad. Sci. USA 84:648-52; PCT Publication No. WO88/09810) or the blood-brain barrier (see, e.g., PCT Publication No. WO89/10134). In addition, the polynucleotides can be modified with hybridization-triggered cleavage agents (see, e.g., Krol et al., 1988, Bio-Techniques 6:958-76) or intercalating agents (see, e.g., Zon, 1988, Pharm. Res. 5:539-49). Furthermore, the polynucleotides can also contain detectable labels such as chemiluminescent, fluorescent, radioactive, and colorimetric labels.

A target “compound” may be a target polynucleotide, polypeptide, other biomolecule, or their derivatives. As used herein, the term “polypeptide” refers to a polymer of amino acids, wherein the α-carboxyl group of one amino acid is joined to the α-amino group of another amino acid by a peptide bond. A protein may comprise one or multiple polypeptides linked together by disulfide bonds. Examples of proteins include, but are not limited to, antibodies, antigens, ligands, receptors, etc. Other biomolecules include, but are not limited to, carbohydrates, lipids, their conjugates, haptens, and analogues thereof. Preferably, a target compound is able to bind a polynucleotide to form a non-covalent polynucleotide-target compound complex.

In a cross-linking reaction, upon exposure to a suitable light source, a photoreactive cross-linking agent is subject to activation and causes a cross-linked product to form between a polynucleotide and a target compound. The polynucleotide participates as an electronic donor to the target compound. The target compound contains a phenolic substituent, e.g., tyrosine, that can be activated to become an acceptor of the electron. In certain embodiments, the polynucleotide, target compound and/or photoreactive cross-linking agent are provided in partially or fully purified forms. In other embodiments, the polynucleotide, target compound and/or photoreactive cross-linking agent are in the form of a complex mixture that may include, for example, aqueous or organic solvent, proteins, lipids, nucleic acids, detergents, particulates, intact cells, as well as other components. The polynucleotide, target compound and photoreactive cross-linking agent may be prepared from naturally occurring sources or through chemical synthesis according to the procedures known in the art. They can also be obtained from vendors if they are commercially available.

The cross-linking reaction is initiated by irradiation with electromagnetic waves (here generally referred to as “light”). The light source is usually selected according to the properties of the photoreactive cross-linking agent. In order to trigger the cross-linking process, the photons must have a certain minimum energy sufficient enough to activate the photoreactive cross-linking agent. Preferably, the photoreactive cross-linking agent is activatable by a visible light for convenience and low cost. For example, Ru(II)bpy₃²⁺ is an efficient photoreactive cross-linking agent with a λₘₐₓ of 452 nm and a molar extinction coefficient of 14,700 M⁻¹. It can be activated by a white light of 380-780 nm. Photolysis of this metal complex in the presence of APS generates Ru(III) and sulfate radical. Ru(III) is a potent one-electron oxidant, and the sulfate radical is a good hydrogen atom abstractor. Without binding by the theory, it is believed that Ru(III) oxidizes residues such as tyrosine, and the sulfate radical plays a key role in forming a stable linkage between two proteins. See, e.g., Fancy and Kodadek, 1999, Proc. Natl. Acad. Sci. USA 96(11):6020-4.

More preferably, the light source delivers as uniform as possible a flow of energy through the desired volume of the material to be cross-linked, and the total energy is high enough for the cross-linking process to complete within as short a time as possible, providing a good, economically viable yield in a production process. As well known in the art, other optics such as condensers and filters may be employed to obtain a light with desired intensity and wavelength.

The method of the invention has broad applicability where a polynucleotide is to be cross-linked to a target compound. For example, it can be used to identify a target compound that binds to a polynucleotide (e.g., where the polynucleotide is known and the target compound is not), or a target polynucleotide that binds to a compound (e.g., where the compound is known and the target polynucleotide is not). A target compound or polynucleotide thus identified may be useful for diagnosis and treatment of diseases, and for development of new compounds for pharmaceutical, medical or industrial purposes.

In one embodiment, the invention provides an assay for screening test compounds that bind to a polynucleotide. In another embodiment, the invention provides an assay for screening test polynucleotides that bind to a compound. The screening of test compounds is described in detail below as an example.

The test compounds can be obtained using any of the numerous approaches in combinatorial library methods known in the art (see, e.g., U.S. Pat. No. 6,689,865), including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic libraries requiring deconvolution; the “one-bead one-compound” libraries; and synthetic libraries produced using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145). Examples of methods for the synthesis of molecular libraries can be found in the art, for example, in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233. Libraries of test compounds may be presented in solutions (e.g., Houghten, 1992, Biotechniques 13:412-21), or on beads (Lam, 1991, Nature 354:82-4), chips (Fodor, 1993, Nature 364:555-6), bacteria (Ladner, U.S. Pat. No. 5,223,409), spores (Ladner, U.S. Pat. No. 5,223,409), plasmids (Cull et al., 1992, Proc. Natl. Acad. Sci. USA 89:1865-9), or phages (Scott and Smith, 1990, Science 249:386-90; Devlin, 1990, Science 249:404-6; Cwirla et al., 1990, Proc. Natl. Acad. Sci. USA 87:6378-82; Felici, 1991, J. Mol. Biol. 222:301-10; and Ladner supra).

In one embodiment, the assay is conducted in a cell-based system. A cell which expresses a target compound is contacted with a polynucleotide and a photoreactive cross-linking agent. After being exposed to a light source, the polynucleotide is cross-linked to the target compound. The cross-linked product is then isolated, and the target compound identified.

In another embodiment, a cell-free system is provided in which a polynucleotide is contacted with a test compound and a photoreactive cross-linking agent. For example, soluble and/or membrane-bound forms of isolated proteins can be used in a cell-free system. When membrane-bound forms of the proteins are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), and N-dodecyl=N,N-dimethyl-3-ammonio-1-propane sulfonate.

To facilitate the isolation of the cross-linked product, the polynucleotide may be coupled, for example, with a radioisotope or enzymatic label such that the cross-linked product can be identified by detecting the labeled polynucleotide in a complex. For instance, the polynucleotide can be labeled with ¹²⁵I, ³⁵S, ¹⁴C, or ³H, either directly or indirectly, and the radioisotope detected by direct counting of radioemmission or by scintillation counting. Alternatively, the polynucleotide can be enzymatically labeled with, for example, horseradish peroxidase, alkaline phosphatase, or luciferase, and the enzymatic label detected by determination of conversion of an appropriate substrate to a product. The signals produced may be detected by a naked eye or by means of a specially designed instrumentation. For example, in one embodiment, a fluorescent signal is recorded with a charged coupled device (CCD) camera. It would be appreciated by those skilled in the art that any detection method may be used as long as it provides consistent and accurate results.

Further, it may be desirable to immobilize either the polynucleotide or its target compound to facilitate separation of the cross-linked complex from uncomplexed form of the target compound or the polynucleotide, and the uncross-linked polynucleotide-target compound complex, as well as to accommodate automation of the assay. The assay can be accomplished in any vessel suitable for containing the reactants. Examples of such vessels include microtiter plates, test tubes, and micro-centrifuge tubes. In one embodiment, the polynucleotide is bound to a substrate. In another embodiment, the target compound is bound to a substrate. Exemplary substrates include Langmuir-Blodgett film, functionalized glass, germanium, silicon, PTFE, polystyrene, gallium arsenide, gold, silver, membrane, nylon, glass bead, magnetic bead, PVP, and microtiter plates. Techniques for attaching polynucleotides to a substrate are well known in the art, including: e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based methods (e.g., as described in PCT US/93/04145). Target compounds can also be attached to a substrate by various methods known in the art. For example, when a target compound is a protein, a fusion protein of glutathione-S-transferase (GST) and the target protein can be adsorbed onto glutathione sepharose beads (Sigma Chemical, St. Louis, Mo.) or glutatilione-derivatized microtiter plates. Other techniques for immobilizing a polynucleotide or a target compound on a substrate include using conjugation of biotin and streptavidin. Biotinylated polynucleotide or target compound can be prepared from biotin-NHS(N-hydroxy-succinimide) using techniques known in the art (e.g., biotinylation kit, Pierce Chemicals, Rockford, Ill.), and immobilized in the wells of streptavidin-coated plates (Pierce Chemical).

The reaction mixture is incubated under conditions sufficient for complex formation and cross-linking. Such conditions are known in the art or can be readily determined without undue experimentation by a skilled artisan. Examples of such conditions are described in Examples 1 and 2 below. Following the cross-linking, the uncross-linked complexes may be disrupted, e.g., under denaturing conditions such as NaOH, urea, SDS and heating. The beads or microtiter plate wells are then washed to remove any unbound components, and the cross-linked complexes detected either directly or indirectly. Detection of the cross-linked complexes anchored on the substrate can be accomplished in a number of ways. Where the previously non-immobilized component is pre-labeled, detection of the label immobilized on the substrate indicates that the cross-linked complexes were formed. Where the previously non-immobilized component is not pre-labeled, an indirect label can be used to detect the cross-linked complexes anchored on the substrate, e.g., using a labeled antibody specific for the immobilized component (the antibody, in turn, can be directly labeled or indirectly labeled with, e.g., a labeled anti-Ig antibody). Alternatively, the cross-linked complexes can be dissociated from the substrate and detected using standard techniques.

Cell-free assays can also be conducted in a liquid phase. In such an assay, the cross-linked complexes are separated from uncomplexed components (including those dissociated from uncross-linked complexes by a denaturing agent) by any of a number of standard techniques, including but not limited to: differential centrifugation (see, for example, Rivas and Minton, 1993, Trends Biochem. Sci. 18:284-7), chromatography (e.g., gel filtration chromatography and ion-exchange chromatography), electrophoresis (see, e.g., Ausubel et al., eds. Current Protocols in Molecular Biology, 1999, J. Wiley: New York.), and immunoprecipitation (see, for example, Ausubel et al., supra). Such resins and chromatographic techniques are known to one skilled in the art (see, e.g., Heegaard, 1998, J. Mol. Recognit. 11:141-8; Hage and Tweed, 1997, J. Chromatogr. B. Biomed. Sci. Appl. 699:499-525).

Once the polynucleotide is cross-linked to its target compound, the cross-linked product can be analyzed by any method well known in the art. For instance, the cross-linked product may be analyzed using affinity interaction, size exclusion, electrophoresis, and a combination thereof. Examples of affinity interactions include, but are not limited to, an immunoaffinity capture process, an immunoaffinity hybridization process, and a combination thereof. Specifically, the immunoaffinity capture process may involve antibody, antigen, aptamer probe arrays, immunoaffinity chromatography, or a combination thereof. Alternatively, the affinity interaction may be a nucleic acid or aptamer microarray-based hybridization process. Examples of size exclusions include, but are not limited to, gel chromatography, high performance size exclusion chromatography, membrane-based size exclusion, and a combination thereof. Examples of electrophoreses include, but are not limited to, gel electrophoresis, capillary electrophoresis, capillary affinity interaction electrophoresis, and a combination thereof. On the other hand, the target compound and the polynucleotide of the cross-linked polynucleotide-target compound may also be identified by mass spectrometry.

The method of the invention can also be used to attach a polynucleotide to a substrate containing a compound. For example, a substrate may be coated with a compound according to the methods known in the art. The coated substrate is then contacted with a polynucleotide and a photoreactive cross-linking agent under conditions that allow the polynucleotide to be cross-linked to the compound.

In one embodiment, arrays are produced by attaching polynucleotides to a substrate containing their partner compounds. Arrays are important tools for gene expression analysis, DNA sequencing, mutation detection, polymorphism screening, linkage analysis, genotyping, and screening for alternative splice variants in gene transcripts. These analyses can provide critical keys to diagnosis, prognosis, and treatment of a variety of diseases in animals, including humans, and plants.

Contacting of the polynucleotides and photoreactive cross-linking agent with the compounds coated on a substrate may be carried out by jet printing, solid or open capillary device contact printing, microfluidic channel printing, silk screening, and printing using devices based upon electrochemical or electromagnetic forces. For example, thermal inkjet printing techniques utilizing commercially available jet printers and piezoelectric microjet printing techniques, as described in U.S. Pat. No. 4,877,745, may be utilized to spot the polynucleotides and the photoreactive cross-linking agent to the coated substrate. A Biomek High Density Replicating Tool (HDRT) (Beckman Coulter, Calif.) may also be used for an automatic gridding. Alternatively, the contacting step may be carried out by manual spotting of the polynucleotides and the photoreactive cross-linking agent onto the coated substrate. Examples of manual spotting include, but are not limited to, manual spotting with a pipettor. It should be understood that the coated substrate may be exposed to the polynucleotides and the photoreactive cross-linking agent by any methods as long as the polynucleotides and the photoreactive cross-linking agent are put in direct contact with the compounds.

Many applications utilizing immobilized polynucleotides require that the polynucleotides be immobilized at site-specific locations on a substrate surface. Accordingly, a plurality of polynucleotides may be placed and cross-linked on the surface of the substrate coated with their partner compounds. In order to prepare ordered arrays of polynucleotides with each polynucleotide located at site-specific locations, a preselected site on the surface of the substrate is exposed to a solution of the desired polynucleotide. This can be accomplished manually by applying an aliquot of the polynucleotide solution to a preselected location on the substrate. Alternatively, thermal inkjet printing techniques utilizing commercially available jet printers and piezoelectric microjet printing techniques, as described in U.S. Pat. No. 4,877,745, can be utilized to spot selected substrate surface sites with selected polynucleotides.

In an alternative embodiment, the polynucleotides may be attached to beads such as magnetic beads. Polynucleotide beads can be used in a wide range of biological assays, for example, binding analysis and affinity chromatography. Use of magnetic beads would facilitate isolation of the polynucleotides, e.g., after hybridization to their complementary sequences.

Magnetic particles may be prepared by precipitating metal salts in base to form fine magnetic metal oxide crystals, and redispersing and washing the crystals in water and in an electrolyte (see, e.g., U.S. Pat. No. 6,569,630). Magnetic separations may be used to collect the crystals between washes if the crystals are superparamagnetic (i.e., do not become permanently magnetized). The crystals may then be coated with a compound capable of binding a polynucleotide. The coated beads are mixed with the polynucleotides and photoreactive cross-linking agent. The cross-linking reaction is triggered by a light, and the polynucleotide is covalently bound to the compound on the beads.

The magnetic particles may come in different sizes suitable for specific applications. Large magnetic particles can respond to weak magnetic fields and magnetic field gradients. However, they tend to settle rapidly, limiting their usefulness for reactions requiring homogeneous conditions. Large particles also have a more limited surface area per weight than smaller particles, so that less material can be coupled to them.

Ferromagnetic beads in general become permanently magnetized in response to magnetic fields. Superparamagnetic beads, in contrast, experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms in diameter; larger crystals generally have a ferromagnetic character. Superparamagnetic particles are usually preferred, as they do not exhibit the magnetic aggregation associated with ferromagnetic particles and permit redispersion and reuse.

The invention also features a kit for cross-linking a polynucleotide to a target compound. The kit contains a polynucleotide and a photoreactive cross-linking agent. When the polynucleotide and photoreactive cross-linking agent are contacted with a target compound, the polynucleotide is cross-linked to the target compound. Optionally, the kit may also include an insert (e.g., a label or printed information) describing use of the photoreactive cross-linking agent to cross-link the polynucleotide to a target compound. In certain embodiments, the kit may include other components such as reagents for detecting the formation of the cross-linked product.

The invention provides a method and a kit for cross-linking a polynucleotide to a target compound through the action of a photo-activatable cross-linking agent. The reaction can be triggered at a desired time and accomplished very rapidly and in high yield. The process can be easily automated, e.g., for A² plate multiplex assays and bead-based assays.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example 1

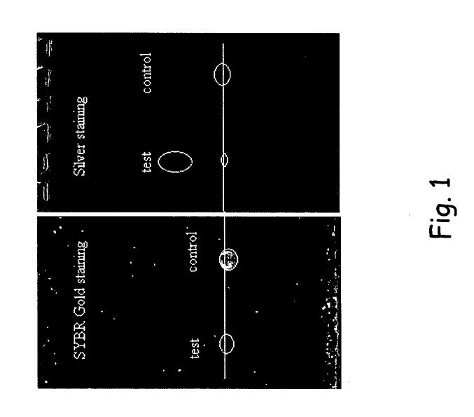

bFGF photoaptamer (GCG AAG GCA CAC CGA GXX CAX AGX AXC CCA, where X=BrdU; SEQ ID NO:1) was mixed with FGFb ligand overnight to achieve maximal aptamer-ligand binding. White light-mediated photolysis of tris-bipyridylruthenium (II) complex was employed to cross-link the aptamer to the ligand in the presence of APS. The solution was then boiled in the presence of urea-SDS to dissociate bound but uncross-linked ligand from the aptamer. The mixture was subsequently applied to a urea slab gel to separate free aptamer from the cross-linked aptamer-ligand complex by electrophoresis. SYBR Gold staining was used to determine the presence of the aptamer, while double-staining with silver stain was used to determine the presence of the ligand. Referring to FIG. 1, the test sample consisted of bFGF photoaptamer and fibroblast growth factor-basic (FGFb) ligand; the control sample consisted of only bFGF photoaptamer. The test and control samples were incubated overnight prior to PICUP (photo-induced cross-linking of unmodified proteins)-like cross-linking. The samples (12 ul/each) were run on a 6% TBE-urea gel. In the left panel, the weaker SYBR Gold staining signal (circled) in the test sample indicates that the aptamer was complexed with the FGFb ligand and therefore was not available to bind SYBR Gold. The Rf values for the aptamer control samples were the same for both silver staining (the right panel) and SYBR Gold staining, while a new band corresponding to the aptamer-FGFb complex was seen with a lower Rf in silver staining of the test sample. Both the test and control samples were subject to denaturing conditions (100° C. for 5 minutes in TBE-urea sample buffer) prior to electrophoresis. As shown in FIG. 1, after cross-linking, the aptamer band disappeared (SYBR Gold staining), whereas a presumed aptamer-ligand complex appeared (silver staining). These results suggest that photo-induced cross-linking occurred between the aptamer and the ligand.

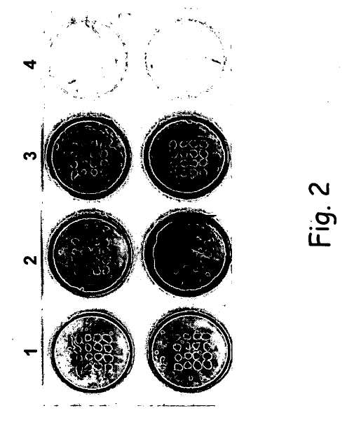

In FIG. 2, FGFb ligand was applied to an array of aptamers and incubated for 48 hours to achieve maximal binding. Photo-induced cross-linking samples (columns 1 and 2) were compared with control samples (columns 3 and 4) that had not been subjected to the photolysis process. Each well of the A² plate (flexible format) was rinsed and the following solutions applied for incubation overnight: columns 1 and 4—1N NaOH, 1% SDS; columns 2 and 3—TBS/Tween buffer. As shown in FIG. 2, NaOH solution effectively dissociated aptamer-ligand that had not been cross-linked (column 4), whereas photo-induced cross-linking resulted in a stable complex (column 1). Fluorescent signals were generated using biotinylated anti-FGF with streptavidin-AP+ELF reagent and captured with a CCD camera.

### Example 2

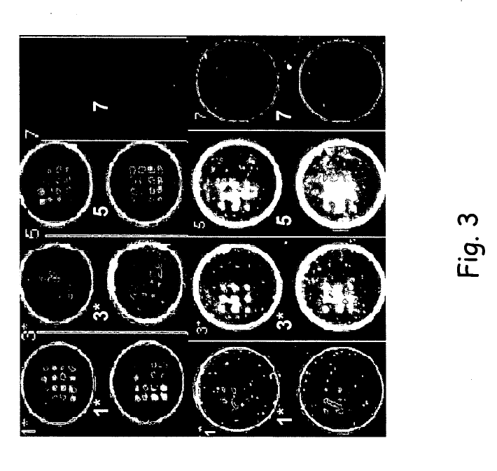

FGFb bindings by bFGF photoaptamer and bFGF native aptamer (GCG AAG GCA CAC CGA GTT CAT AGT ATC CCA; SEQ ID NO:2) were compared. Referring to FIG. 3, * denotes PICUP-like cross-linking (the left two columns), while the remaining wells represent non-covalent ligand capture (the right two columns). Samples in plates 1* and 7 were treated with 1N NaOH, 1% SDS to remove the non-covalently bound ligand. As shown in FIG. 3, PICUP-like cross-linking between FGFb and the unmodified aptamer (columns 1* and 7 in the lower panel) was reduced after treatment with denaturants compared to the untreated controls (columns 3* and 5 in the lower panel) and the BrdU-modified aptamer (column 1* in the upper panel). These results indicate that, although the native aptamer binds as tightly to the FGFb ligand as the BrdU-modified analog, there are fewer sites in the native aptamer for covalent cross-linking than in the BrdU-modified analog.

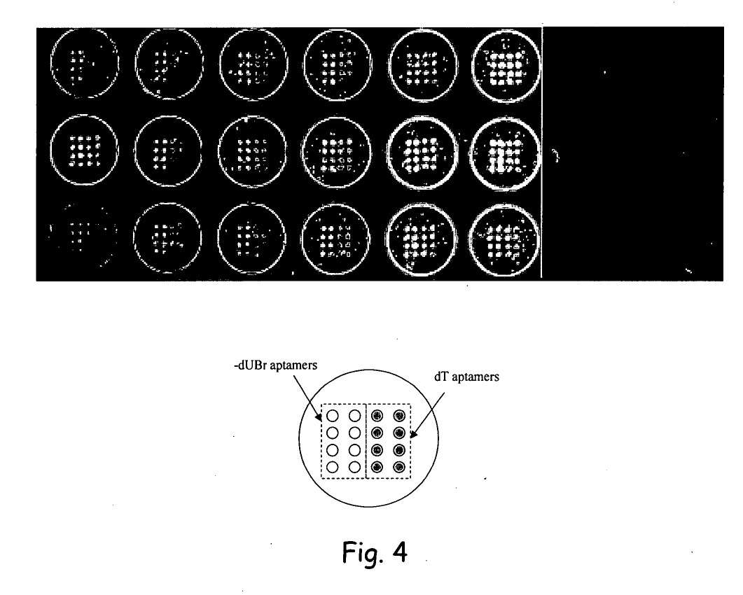

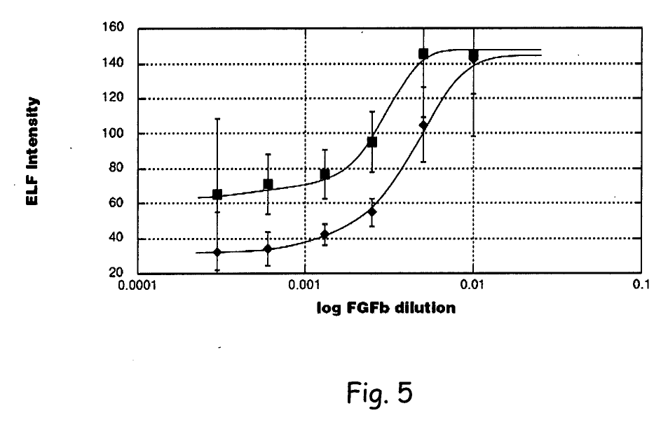

This hypothesis was tested by titrating increasing concentrations of FGFb against the two aptamers and plotting binding curves. Referring to FIG. 4, in the left panel, the two left hand side columns of each array were printed with bFGF photoaptamer, while the two right hand side columns were printed with bFGF native aptamer. The eighteen arrays in the left panel were cross-linked using a PICUP-like protocol, whereas the six arrays in the right panel represent non-covalent binding. All wells were treated with 1N NaOH, 1% SDS to strip the non-covalently bound ligand. The displacement of the bFGF native aptamer binding curve (diamonds) to the right of the bFGF photoaptamer curve (squares) confirmed that the cross-linking avidity of the bFGF native aptamer is lower than its BrdU-modified bFGF photoaptamer analog (FIG. 5).

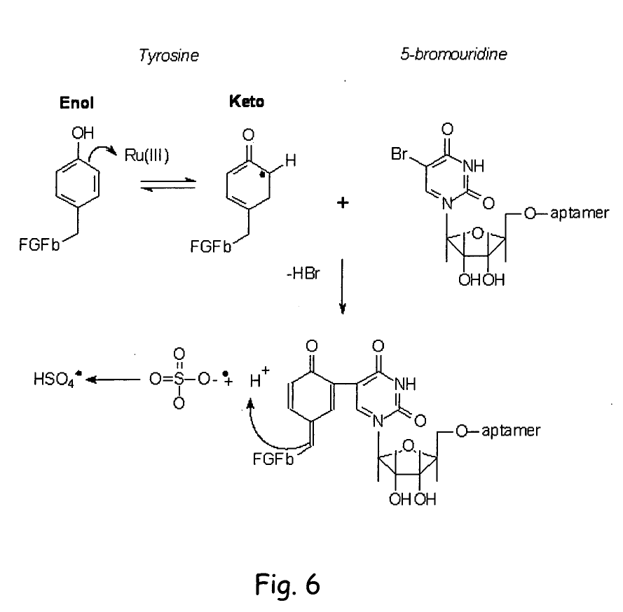

Without binding by the theory, assuming that PICUP-like cross-linking occurs through common adenyl and cytosyl amino groups on both aptamers reacting with tyrosine side chains (Fancy and Kodadek, 1999, Proc. Natl. Acad. Sci. USA 96(11):6020-4), another mechanism would be required to increase the number of tyrosine binding sites on the BrdU-modified bFGF photoaptamer. It is proposed that, in the bFGF photoaptamer, the BrdU bases substituting for thymines can also react with tyrosines via a PICUP-like mechanism (FIG. 6). Thus, bFGF photoaptamer would have five additional BrdU cross-linking sites compared to the bFGF native aptamer, which would increase its avidity in the PICUP-like cross-linking reaction.

While the foregoing has been described in considerable detail and in terms of preferred embodiments, these are not to be construed as limitations on the disclosure or claims to follow. Modifications and changes that are within the purview of those skilled in the art are intended to fall within the scope of the following claims. All literatures cited herein are incorporated by reference in their entirety.

What is claimed is:

1. A method of cross-linking a polynucleotide to a target compound, comprising
providing a polynucleotide and a target compound; and
contacting the polynucleotide and the target compound with a photoreactive cross-linking agent under conditions that allow cross-linking of the polynucleotide to the target compound.

2. The method of claim 1, wherein the target compound is an electron acceptor.

3. The method of claim 1, wherein the polynucleotide is an electron donor.

4. The method of claim 1, wherein the target compound is a modified electron acceptor.

5. The method of claim 1, wherein the polynucleotide is to a modified electron donor.

6. The method of claim 1, wherein the polynucleotide donates electrons to the target compound.

7. The method of claim 1, wherein the target compound is a polypeptide.

8. The method of claim 7, wherein the polynucleotide contains BrdU.

9. The method of claim 8, wherein the contacting step further comprising activating the cross-linking agent with a visible light.

10. The method of claim 9, wherein the cross-linking agent is Ru(II)bpy₃²⁺.

11. The method of claim 10, wherein the polynucleotide is cross-linked to the target compound in the presence of APS.

12. The method of claim 1, wherein the target compound is a target polynucleotide.

13. The method of claim 1, wherein the polynucleotide contains BrdU.

14. The method of claim 1, wherein the contacting step further comprising activating the cross-linking agent with a visible light.

15. The method of claim 1, wherein the cross-linking agent is Ru(II)bpy₃²⁺.

16. The method of claim 15, wherein the polynucleotide is cross-linked to the target compound in the presence of APS.

17. The method of claim 1, wherein the cross-linked polynucleotide-target compound is analyzed by affinity interaction, size exclusion, electrophoresis, or a combination thereof.

18. The method of claim 17, wherein the affinity interaction is an immunoaffinity capture process, an immunoaffinity hybridization process, a nucleic acid or aptamer microarray-based hybridization process, or a combination thereof.

19. The method of claim 18, wherein the immunoaffinity capture process comprises antibody, antigen, aptamer probe arrays, immunoaffinity chromatography, or a combination thereof.

20. The method of claim 17, wherein the size exclusion is gel chromatography, high performance size exclusion chromatography, membrane-based size exclusion, or a combination thereof.

21. The method of claim 17, wherein the electrophoresis is gel electrophoresis, capillary electrophoresis, capillary affinity interaction electrophoresis, or a combination thereof.

22. The method of claim 1, wherein the target compound and the polynucleotide of the cross-linked polynucleotide-target compound are identified by mass spectrometry.

23. A method of identifying a target compound that binds a compound, comprising
providing a compound and a test compound, either of which is a polynucleotide;
contacting the compound and the test compound with a photoreactive cross-linking agent in a system; and
determining whether the compound is cross-linked to the test compound, wherein cross-linking of the compound to the test compound indicates that the test compound is a target compound that binds the compound.

24. The method of claim 23, wherein either the compound or the test compound is a polypeptide.

25. The method of claim 23, wherein both the compound and the test compound are polynucleotides.

26. The method of claim 23, wherein the system is a cell-based system or a cell free system.

27. The method of claim 23, wherein either the compound or the test compound is immobilized to a substrate.

28. The method of claim 27, wherein the substrate is a microtiter plate or a bead.

29. The method of claim 23, wherein the cross-linked compound-target compound is analyzed by affinity interaction, size exclusion, electrophoresis, or a combination thereof.

30. The method of claim 29, wherein the affinity interaction is an immunoaffinity capture process, an immunoaffinity hybridization process, a nucleic acid or aptamer microarray-based hybridization process, or a combination thereof.

31. The method of claim 30, wherein the immunoaffinity capture process comprises antibody, antigen, aptamer probe arrays, immunoaffinity chromatography, or a combination thereof.

32. The method of claim 29, wherein the size exclusion is gel chromatography, high performance size exclusion chromatography, membrane-based size exclusion, or a combination thereof.

33. The method of claim 29, wherein the electrophoresis is gel electrophoresis, capillary electrophoresis, capillary affinity interaction electrophoresis, or a combination thereof.

34. The method of claim 23, wherein the target compound and the compound of the cross-linked compound-target compound are identified by mass spectrometry.

35. A method of attaching a polynucleotide to a substrate, comprising
providing a polynucleotide;
providing a substrate containing a compound that binds the polynucleotide; and
contacting the polynucleotide and the compound with a photoreactive cross-linking agent under conditions that allow cross-linking of the polynucleotide to the compound.

36. The method of claim 35, wherein the compound is a polypeptide.

37. The method of claim 35, wherein the substrate is a microtiter plate or a bead.

38. The method of claim 37, wherein the substrate is a magnetic bead.

39. A kit comprising a polynucleotide and a photoreactive cross-linking agent for cross-linking the polynucleotide to a target compound.

40. The kit of claim 39, wherein the target compound is a polypeptide.

41. The kit of claim 40, wherein the polynucleotide contains BrdU.

42. The kit of claim 41, wherein the cross-linking agent is capable of being activated by a visible light.

43. The kit of claim 42, wherein the cross-linking agent is Ru(II)bpy₃²⁺.

44. The kit of claim 43, wherein the polynucleotide is capable of being cross-linked to the target compound in the presence of APS.

45. The kit of claim 39, wherein the target compound is a target polynucleotide.

46. The kit of claim 39, wherein the polynucleotide contains BrdU.

47. The kit of claim 39, wherein the cross-linking agent is capable of being activated by a visible light.

48. The kit of claim 39, wherein the cross-linking agent is Ru(II)bpy₃²⁺.

49. The kit of claim 48, wherein the polynucleotide is capable of being cross-linked to the target compound in the presence of APS.


SEQUENCE LISTING (2 sequences)

SEQ ID NO: 1 - DNA, 30 bp, Artificial sequence
  Synthetic oligonucleotide
gcgaaggcacaccgagnncanagnanccca

SEQ ID NO: 2 - DNA, 30 bp, Artificial sequence
  Synthetic oligonucleotide
gcgaaggcacaccgagttcatagtatccca